(19) United States
(12) Patent Application Publication
(10) Pub. No.: US 2011/0166580 A1
(43) Pub. Date: Jul. 7, 2011

(54) Bone Compression And Distraction Apparatus

(76) Inventor: Adam John Keilen, Kalamazoo, MI (US)

(21) Appl. No.: 12/905,310

(22) Filed: Oct. 15, 2010

Related U.S. Application Data
(60) Provisional application No. 61/279,049, filed on Oct. 15, 2009.

(51) Int. Cl. A61B 17/60

(52) U.S. Cl. 606/90

(57) ABSTRACT

A bone compression and distraction apparatus comprising a body, a worm screw and left and right wire attachments. The body defines an elongated tubular member with a first end and a second end. The worm screw is positioned within the body. The worm screw has right hand threads extending from a central region to a first end thereof and left hand threads extending from the central region to a second end thereof. The right wire attachment threadedly engages the right hand threads. The left wire attachment threadedly engages the left hand threads. Rotation of the worm screw either directs the right and left wire attachments toward each other or away from each other.

### CROSS-REFERENCE TO RELATED APPLICATION

This application claims priority from U.S. Provisional Pat. App. Ser. No. 61/279,049 filed Oct. 15, 2010, entitled Bone Compression and Distraction Apparatus, the entire specification of which is hereby incorporated by reference.

### BACKGROUND OF THE DISCLOSURE

1. Field of the Disclosure

The present disclosure is directed in general to the technical field of medical surgery. More particularly, the present invention is directed to bone compression and/or distraction during surgical procedures.

2. Background Art

Bone compression instruments and bone distraction instruments are known in the art. Instruments that allow both compression and distraction save time by allowing the surgeon to perform both tasks without having to interchange tools during the procedure.

While the devices are known, many existing bone compression/distraction instruments have several short comings. Many are excessively large, which may preclude their use for smaller joint, maxillofacial, podiatric, or hand surgeries. Smaller existing instruments are often composed of complex assemblies and require a large number parts, which increases the cost and can complicate cleaning of the instrument after each procedure. Furthermore, certain existing instruments are often disposable, which increases the cost of care.

### SUMMARY OF THE INVENTION

The present invention is a bone compression and distraction apparatus. The apparatus, among other features, allows the wire attachments to travel simultaneously, in opposing directions, along a worm screw having two thread configurations (left and right). Such a configuration allows fewer parts, easier assembly, and improved cleaning over previous instruments.

### BRIEF DESCRIPTION OF THE DRAWINGS

The disclosure will now be described with reference to the drawings wherein:

FIG. 1 is a perspective view of a Body of the apparatus of the present disclosure;

FIG. 2 is a perspective view of a Closed End Cap of the apparatus of the present disclosure;

FIG. 3 is a perspective view of a Open End Cap of the apparatus of the present disclosure;

FIG. 4 is a perspective view of a Worm Screw of the apparatus of the present disclosure;

FIG. 5 is a perspective view of a Left Wire Attachment of the apparatus of the present disclosure;

FIG. 6 is a perspective view of a Right Wire Attachment of the apparatus of the present disclosure;

FIG. 7 is a perspective view of a Bushing of the apparatus of the present disclosure;

FIG. 8 is a perspective view of a Pin of the apparatus of the present disclosure;

FIG. 10 is an exploded perspective view of the Compression & Distraction apparatus of the present disclosure; and

FIG. 9 is a perspective view of the Compression & Distraction apparatus of the present disclosure without wires or attached ratchet; and

### DETAILED DESCRIPTION OF THE INVENTION

While this invention is susceptible of embodiment in many different forms, there is shown in the drawings and described herein in detail a specific embodiment with the understanding that the present disclosure is to be considered as an exemplification and is not intended to be limited to the embodiment illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

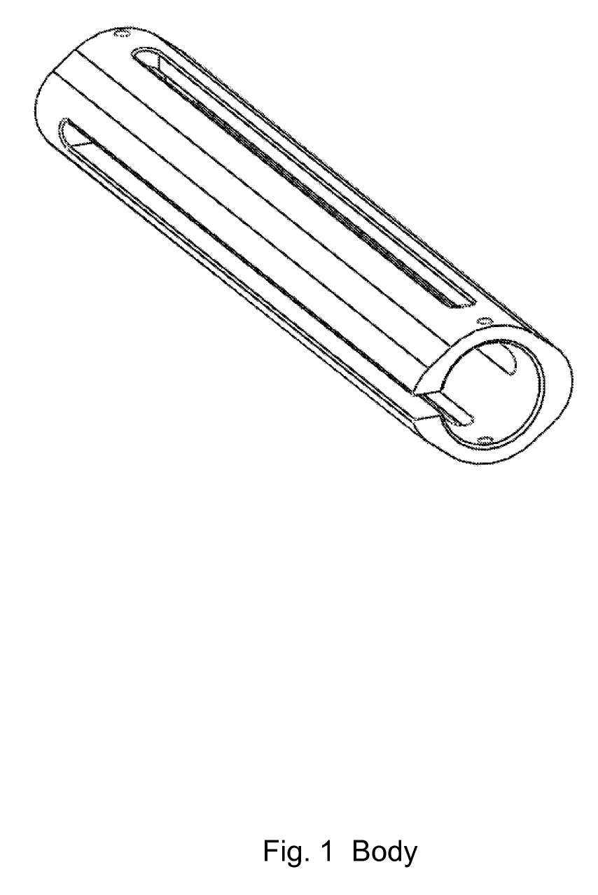

Referring now to the invention in more detail, in FIG. 1 there is shown a Body 10. The Body 10 has a cylindrical hole passing through the entire part axis, thereby defining openings on both ends. The profile of the Body 10 is preferably a parallelogram profile having rounded corners, although other shapes are contemplated. Body 10 has an assembly slot on one rounded corner that is open on one end and closed on the opposite end. This slot allows for assembly and cleaning. Body 10 has a plurality of additional slots, such as the three additional slots on the remaining rounded corners that are closed on both ends. These three slots allow for cleaning of the instrument. Body 10 also has two pin holes on each end, passing transversely therethrough to assist with the assembly of the apparatus.

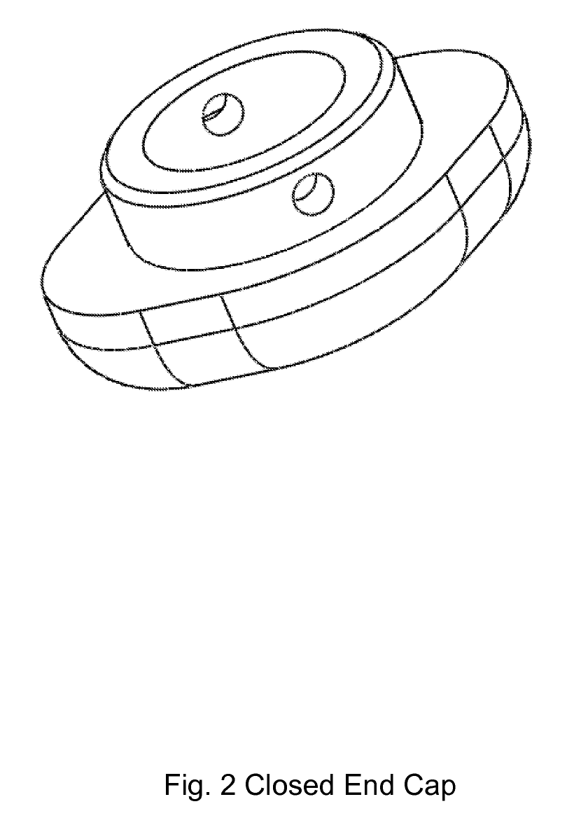

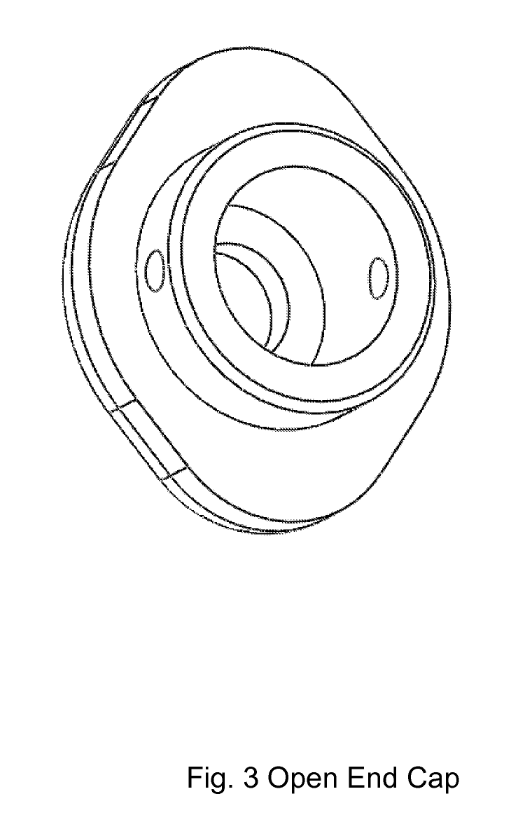

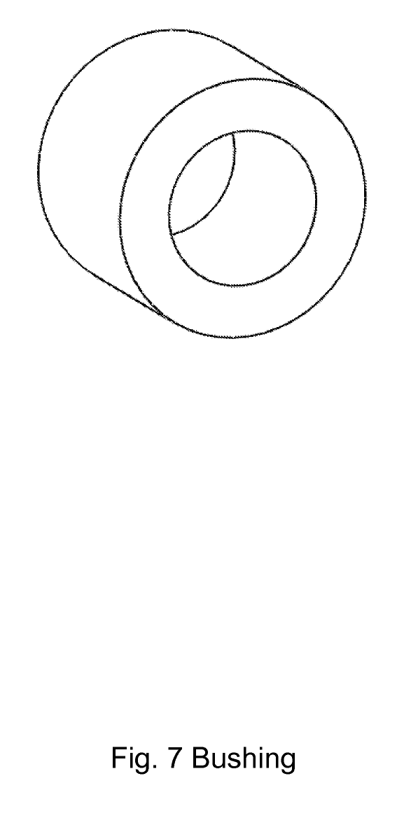

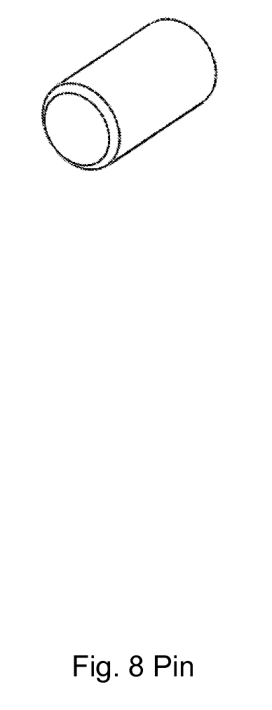

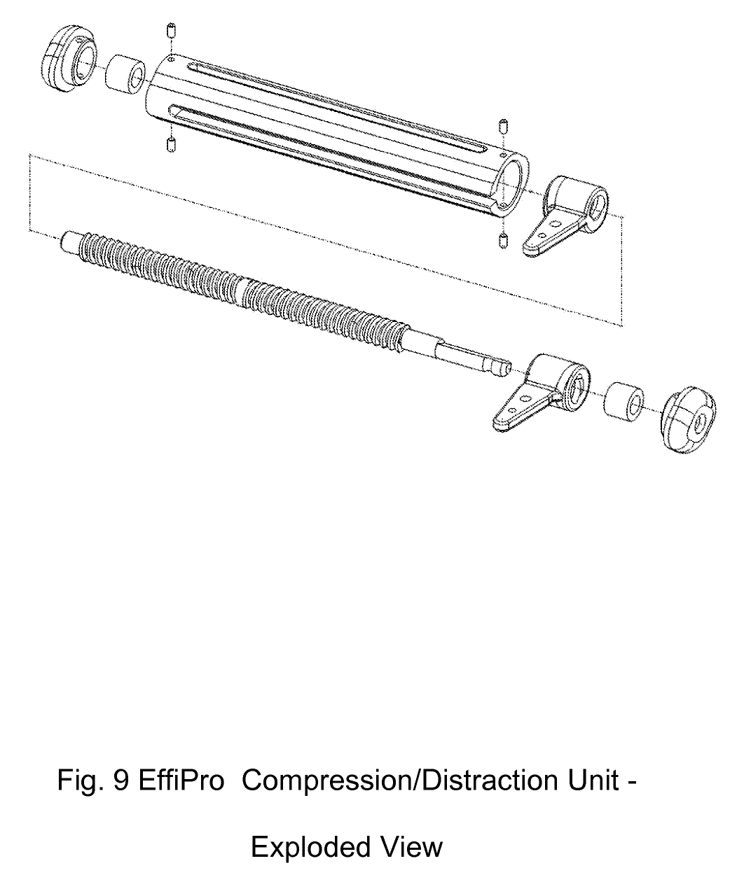

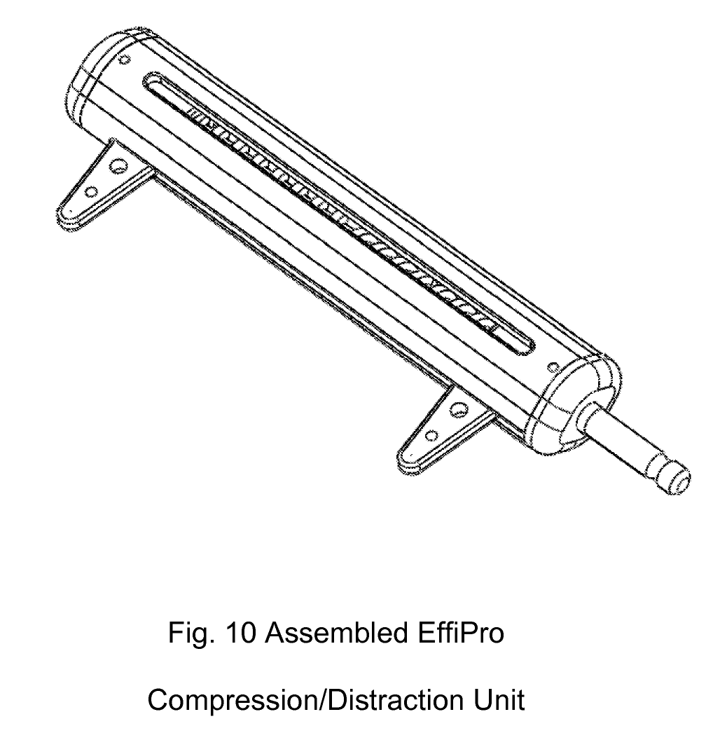

In further detail, the Bushing 22 (FIG. 7) is pressed into the Closed End Cap 12 (FIG. 2), and this subassembly is positioned on the end of the Body 10 toward the closed end of the assembly slot. The Closed End Cap 12 has two blind pin holes to allow assembly. This subassembly is positioned to align the holes in the Closed End Cap 12 with the holes in the Body 10. Once aligned, the Pin 24 is pressed into each hole to attach the Closed End Cap 12 to the Body 10.

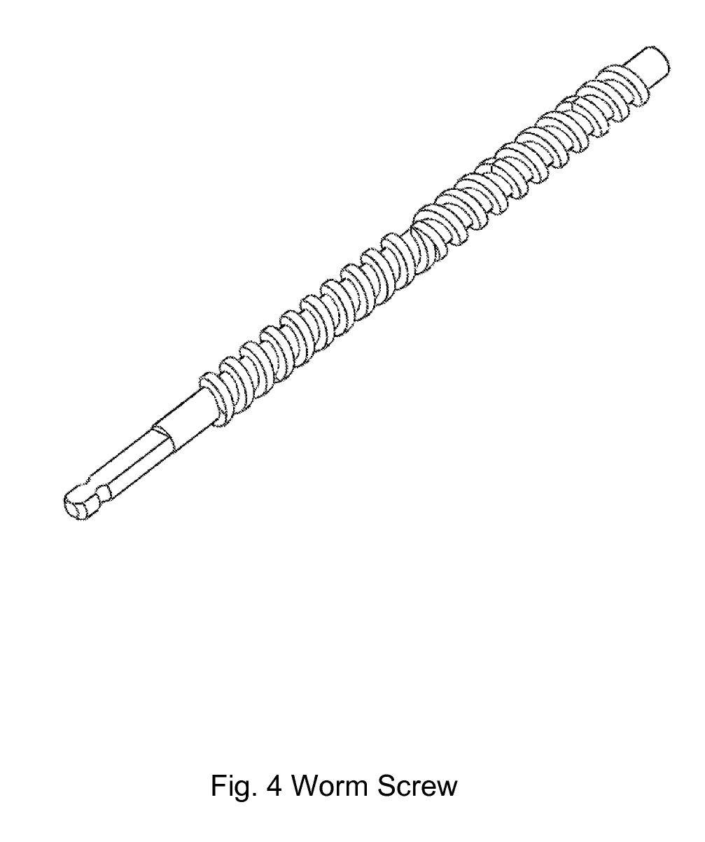

In more detail, the Worm Screw 16 (FIG. 4) is designed with a right hand and left hand stub Acme thread. Each thread starts on opposite ends of the Worm Screw 16 and meets in the center. Located on the end of the Worm Screw 16, having the right hand Acme thread, is a drive feature with a machined flat (FIG. 4). This flat is for receiving a ratchet device.

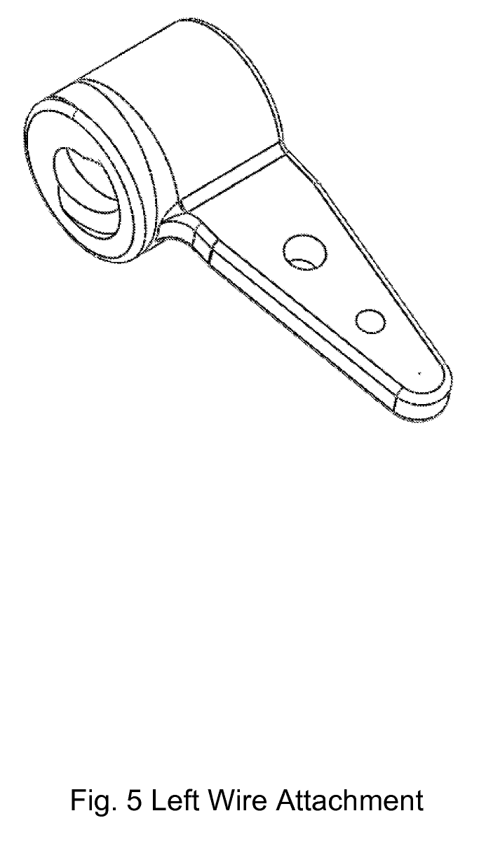

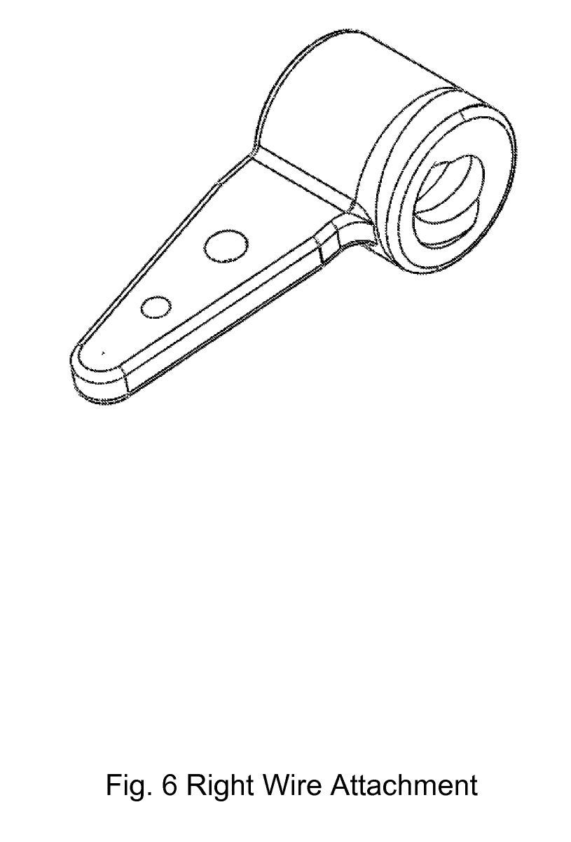

In further detail, the Right Wire Attachment 20 (FIG. 6) is threaded onto the Worm Screw 16 on the side having the right hand thread. This is the end having the ratchet drive feature. The Left Wire Attachment 18 (FIG. 5) is threaded onto the Worm Screw 16 on the side having the left hand thread. Each Wire Attachment is threaded until they meet and stop in the center of the Worm Screw 16.

In more detail, the Worm Screw 16, having the Wire Attachments installed, is assembled to the Body 10 such that the Wire Attachments slide through the assembly slot in the Body 10. The drive feature of the Work Screw 16 resides on the open end of this assembly. The end of the Worm Screw 16 slides into the Bushing 22 located in the Closed End Cap 12.

In further detail, the Bushing 22 (FIG. 7) is pressed into the Open End Cap 14 (FIG. 3), and this subassembly is positioned over the drive feature of the Worm Screw 16. The Open End Cap 14 has two blind pin holes to allow assembly. This subassembly is positioned to align the holes in the Open End Cap 14 with the holes in the Body 10. Once aligned, the Pin 24 is pressed into each hole to attach the Open End Cap 14 to the Body 10. This completes the assembly of the instrument.

In further detail, each Wire Attachment (Right Wire Attachment 20 and Left Wire Attachment 18) has a series of three through holes; each of specific diameter. Each hole is intended to receive a wire of specific diameter. The wire passes through the hole in the Right Wire Attachment 20 and the wire is anchored into the bone. Another wire passes through the hole in the Left Wire Attachment 18 and is anchored in the bone. As the Worm Screw 16 is actuated, the Wire Attachments transmit force into the wires and either compress, or distract, the bone joint. As will be understood the Wire Attachments traverse along a Worm Screw in opposing directions. Such a configuration allows both Wire Attachments to meet in the center of the instrument allowing ease of cleaning.

In further detail, a ratchet can be placed on the drive feature of the Worm Screw 16. The ratchet is used to turn the Worm Screw 16, causing the Right Wire Attachment 20 and Left Wire Attachment 18 to move in opposing directions (either diverging or converging relative to one another). It will be understood that the ratchet that can remain attached to the instrument throughout an entire surgical procedure.

The overall dimensions of the assembled Body 10 is approximately 0.62×0.75×4.12 inches. The overall dimensional envelope of the assembled instrument, considering the drive feature on the Worm Screw 16 and the Wire Attachments is approximately 0.62×1.42×4.9 inches. These dimensions do not include the ratchet. And, the invention is not limited to any particular dimensions set forth above. However, it will be appreciated that such a small size allows for the successful use of the apparatus for procedures on the smallest joints.

The construction details of the invention are set forth below. It will be understood that the component and the materials from which they are formed can be varied within the scope of the present invention. They are not to be deemed limiting. The Body 10 is constructed of a drawn aluminum alloy that is anodized. The Open End Cap 14 and Closed End Cap 12 are also constructed from an anodized aluminum alloy. The Worm Screw 16 is constructed from a stainless steel alloy that is coated to prevent corrosion and galling. The Right Wire Attachment 20 and Left Wire Attachment 18 are constructed from a stainless steel alloy and treated to prevent corrosion and galling. The Bushing 22 is constructed from a bronze alloy to prevent galling and mitigate friction. The Pin 24 is constructed from a stainless steel alloy. Advantageously, the present apparatus is formed from a small number of components.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

What is claimed is:

1. A bone compression and distraction apparatus comprising:
a body defining an elongated tubular member with a first end and a second end;
a worm screw positioned within the body, the worm screw having right hand threads extending from a central region to a first end thereof and left hand threads extending from the central region to a second end thereof;
a right wire attachment threadedly engaging the right hand threads; and
a left wire attachment threadedly engaging the left hand threads,
Whereupon rotation of the worm screw either directs the right and left wire attachments toward each other or away from each other.

2. The bone compression and distraction apparatus of claim 1 wherein the worm screw further includes a ratchet receiving region positioned at the central region between the left and right hand threads.
